(19) United States
(12) Patent Application Publication
(10) Pub. No.: US 2006/0134714 A1
(43) Pub. Date: Jun. 22, 2006

(54) Detection and identification of peptide and protein modifications

(76) Inventors: Narayan Sundararajan, San Francisco, CA (US); Lei Sun, Santa Clara, CA (US); Xing Su, Cupertino, CA (US); Mineo Yamakawa, Campbell, CA (US); Zhang Jingwu, San Jose, CA (US); Selena Chan, San Jose, CA (US); Andrew Berlin, San Jose, CA (US); Tae-Woong Koo, Cupertino, CA (US); Mark Roth, Seattle, WA (US); Phil Gafken, Seattle, WA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/202,862

(22) Filed: Aug. 11, 2005

Related U.S. Application Data
(63) Continuation-in-part of application No. 10/919,699, filed on Aug. 16, 2004.
(60) Provisional application No. 60/587,334, filed on Jul. 12, 2004.

(51) Int. Cl. C12Q 1/48; C12Q 1/34

(52) U.S. Cl. 435/15; 435/18

(57) ABSTRACT

Embodiments of the present invention provide devices and methods for detecting, identifying, distinguishing, and quantifying modification states of proteins and peptides using Surface Enhanced Raman (SERS) and Raman spectroscopy. Applications of embodiments of the present invention include, for example, proteome wide modification profiling and analyses with applications in disease diagnosis, prognosis and drug efficacy studies, enzymatic activity profiling and assays.

### CROSS-REFERENCE TO RELATED APPLICATIONS

The present application claims the benefit under 35 U.S.C. § 119(e) of U.S. Provisional Patent Application No. 60/587,334, filed Jul. 12, 2004, and the benefit of U.S. application Ser. No. 10/919,699, filed Aug. 16, 2004, the disclosures of which are incorporated herein by reference.

### FIELD OF THE INVENTION

Embodiments of the present invention relate generally to the use of Raman spectroscopy for detecting, distinguishing, quantifying, and identifying modifications to and derivatives of amino acids, peptides, and proteins.

### BACKGROUND OF THE INVENTION

Post-translational modifications (PTMs) are believed to play an important role in the biological activity of proteins. Post-translational modifications are chemical processing events that cleave or add modifying groups to proteins for the purpose of modulating precise regulatory functions in a cell. Over 200 different types of PTMs have been described (R. G. Krishna, F. Wold, in PROTEINS: Analysis & Design, Academic Press, San Diego, 121 (1998)) and PTMs such as acetylation (S. K. Kurdistani, S. Tavazoie, M. Grunstein, Cell, 117, 721-733 (2004)), methylation (T. Kouzarides, Curr. Opin. Genet. Dev., 12, 198-209 (2002)), phosphorylation (P. Cohen, Trends Biochem. Sci. 25, 596-601 (2000)), ubiquitination (P. Tyers, P. Jorgensen, Curr. Opin. Genet. Dev. 10, 54-64 (2000)), and others play key roles in the regulation of gene expression, protein turnover, signaling cascades, intracellular trafficking, and cellular structure.

In the past, mass spectrometry (MS) has been a favored approach for proteome-wide PTM profiling due to its sensitivity for measuring and locating molecular weight changes in proteins and peptides. However, some modifications such as acetylation and trimethylation of lysine (both have nominal mass increases of 42 Da) and phosphorylation and sulfation of tyrosine (both have a nominal mass increases of 80 Da) require expensive, high-resolution mass spectrometers or require mass spectrometry analysis schemes that are not conducive to high-throughput analyses. Also, modifications such as phosphorylation, sulfation, and glycosylation are unstable during tandem mass spectrometry experiments making identification and positional information difficult to obtain. In few cases, quantification of protein expression and modifications using mass spectrometry has been performed using stable isotope labeling techniques. See, for example, S. P. Gygi et al., Nature Biotechnology, 17, 994 (1999) and X. Zhang, Q. K. Jin, S. A. Carr, S. A. & RS., Rapid Commun. Mass Spectrom. 16, 2325-32 (2002).

Surface-enhanced Raman spectroscopy (SERS) is a sensitive method for chemical analysis. A Raman spectrum, similar to an infrared spectrum, consists of a wavelength distribution of bands corresponding to molecular vibrations specific to the sample being analyzed (the analyte). Raman spectroscopy probes vibrational modes of a molecule and the resulting spectrum, similar to an infrared spectrum, is fingerprint-like in nature. As compared to the fluorescent spectrum of a molecule which normally has a single peak exhibiting a half peak width of tens of nanometers to hundreds of nanometers, a Raman spectrum has multiple structure-related peaks with half peak widths as small as a few nanometers.

To obtain a Raman spectrum, typically a beam from a light source, such as a laser, is focused on the sample generating inelastically scattered radiation which is optically collected and directed into a wavelength-dispersive spectrometer. Although Raman scattering is a relatively low probability event, SERS can be used to enhance signal intensity in the resulting vibrational spectrum. Enhancement techniques make it possible to obtain an approximately 10⁶ to 10¹⁴ fold Raman signal enhancement. Typically, a surface-enhanced Raman spectrum is obtained by adsorbing a target analyte onto a metal surface. The intensity of the resulting enhancement is dependent on many factors, including the morphology of the metal surface. Enhancements are achieved, in part, through interaction of the adsorbed analyte with an enhanced electromagnetic field produced at the surface of the metal.

### BRIEF DESCRIPTION OF THE DRAWINGS

FIG. 1 is a schematic diagram illustrating steps for protein profiling using SERS or Raman spectroscopy. Optionally, protein profiling may also include mass spectrometry.

FIGS. 2A and 2B illustrate a use of SERS to detect peptide modifications. In FIG. 2A, a substrate containing an array having a multiplexity of peptides at different locations is allowed to interact with a sample of biologic origin (containing, for example, enzymes or cell lysates), and SERS is performed before and after the interaction. In FIG. 2B, a peptide array is made from a digested set of proteins or biofluids deposited on a substrate, selected enzymes are reacted with the peptides of the array, and SERS is performed before and after the enzymatic interaction.

FIG. 3 shows the SERS spectrum of an unmodified peptide (P) (sequence: ⁹KSTGGKAPR) with notations regarding the chemical bonding information that can be derived from the peaks (spectrum was taken at a peptide concentration of 9 ng/μl).

FIG. 4 shows SERS spectra of unmodified and modified peptides (K9 peptide of the histone H3.3 of drosophila): ⁹KSTGGKAPR (P), ⁹K(trimethylated)STGGKAPR (P-9Me3), and ⁹K(acetylated)STGGKAPR (P-9Ac). Spectra were taken a concentration of 9 ng/μl each. The spectra were arbitrarily offset along the y-axis for clarity.

FIG. 5 shows the detection of very low concentrations of trimethylated peptide, P-9Me3. The spectra were arbitrarily offset along the y-axis for clarity. Arrows indicate strong spectral features that are present at all concentrations.

FIGS. 6A and 6B illustrate positional dependence in SERS spectra for two different protein modifications: trimethylation and phosphorylation. In FIG. 6A, the upper line illustrates the SERS spectrum of a peptide that has been trimethylated at a lysine located in the middle of the peptide chain (⁹KSTGG¹⁴K(trimethylated)APR) (P-14Me3), and the bottom line illustrates the SERS spectrum of a peptide having the same sequence that has been trimethylated at the lysine located at the N-terminus of the peptide (⁹K(trimethylated)STGGKAPR) (P-9Me3). Spectra were taken at concentrations of 9 ng/μL and arbitrarily offset along the y-axis. In FIG. 6B, the upper line illustrates the SERS spectrum of a peptide that has been phosphorylated at a threonine (⁹KS ¹¹T(phosphorylated)GGKAPR) (P-11P) and the bottom line illustrates the SERS spectrum of a peptide that has been phosphorylated at a serine (⁹K¹⁰S(phosphorylated)TGGKAPR) (P-10P). Data represents spectra obtained from phosphorylated peptides from a single source. Spectra were taken at concentrations of 90 ng/μL and arbitrarily offset along the y-axis.

FIG. 7 shows a graph of the ratio of intensities of peaks at 744 cm⁻¹ (trimethyl) and 1655 cm⁻¹ (Amide I) wave numbers plotted for the two peptides P-9Me3 and P-14Me3. Fifty spectra (having an accumulation time of 1 s) were collected for each peptide and the peak intensities at 744 cm⁻¹ and 1655 cm⁻¹ were calculated for each spectrum. The averages for the ratios of the intensities for the peptides P-9Me3 and P-14Me3 were 2.499 and 1.644 with standard deviations of 0.0586 and 0.0437, respectively.

FIGS. 8A and 8B provide SERS spectra of the unmodified peptide (⁹KSTGGKAPR) and a ubiquitin analog (⁹K(Gly-Gly)STGGKAPR), respectively. Spectra were taken at concentrations of 90 ng/μL.

FIG. 9A provides SERS spectra of P-9Me2 (⁹K(dimethylated)STGGKAPR) and P-9Me3 (⁹K(trimethylated)STGGKAPR) peptide mixtures in which concentration of P-9Me3 varied from 0% to 100%. The total concentration of the mixture was 70.0 ng/uL. FIG. 9B shows the quantification of modification in mixtures of 9-trimethylated peptide P-9Me3, ⁹K(trimethylated)STGGKAPR and 9-dimethylated peptide P-9Me2, ⁹K(dimethylated)STGGKAPR. The Y-axis represents the ratio of intensities of peaks at 744 cm⁻¹ and 1655 cm⁻¹ from the SERS spectra of different concentration % mixtures. The X-axis represents the % concentration of 9-trimethylated peptide P-9Me3 in the mixture.

FIG. 10 shows a map of the N-terminal tail of Histone H3 and indicates the biological significance of illustrated posttranslational modifications.

FIGS. 11A and 11B show SERS spectra obtained from different unmodified and corresponding trimethylated peptides, respectively, from the N-terminal tail of Histone H3. The sequences for the peptides shown are: ³TKQTAR for the spectra labeled P3-8, ¹⁸KQLATKAAR for the spectra labeled P18-26, and ²⁷KSAPSTGGVKKPHR for the spectra labeled P27-40. Spectra were taken at concentrations of 90 ng/μL.

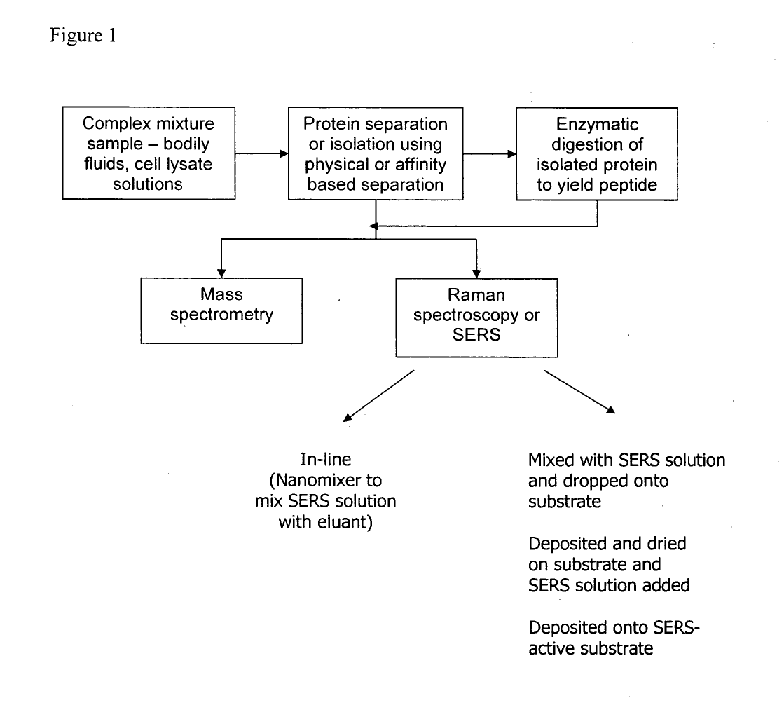

FIG. 1 2A shows an HPLC (high pressure liquid chromatography) chromatogram of digested Histone H3 using a C18 column. FIG. 12B shows MALDI-TOF (matrix-assisted laser desorption ionization—time of flight) mass spectrum of Fraction 2 from the HPLC chromatogram of FIG. 12A. FIG. 12C shows the SERS spectra of Fraction 2 from the HPLC chromatogram of FIG. 12A from digested and separated Histone H3 and synthesized trimethylated peptide (P-9Me3).

FIG. 13 shows SERS spectra of peptide P-9Ac (⁹K_{ac}STGGKAPR) at different incubation times of sample with the colloidal silver solution before addition of lithium chloride to induce aggregation.

FIG. 14A shows a raw sample spectrum of the unmodified peptide P (⁹KSTGGKAPR). Background from the spectra was subtracted by fitting an arbitrary linear baseline. FIG. 14B shows how intensities of peaks were calculated directly from the raw spectra by calculating the distance between the apex of the peak area and the midpoint of the base points of the peak area.

FIG. 15 schematically describes a Raman spectrometer that can be used for SERS measurements.

### DETAILED DESCRIPTION OF THE INVENTION

A variety of modifications to the amino acid building blocks that make up a peptide or a protein are possible, such as for example, dimethylation, trimethylation, acetylation, phosphorylation, ubiquination, palmitoylation, glycosylation, lipidation, sulfation, and nitrosylation. (See also, for example, “Proteomic analysis of post-translational modifications”, Mann et al., Nature Biotechnology, 21:255 (2003)). Embodiments of the present invention provide the ability to detect modification(s) to the amino acids in a peptide or protein at low concentrations, and also to distinguish, identify, and quantify them based on spectral signatures. Detection is possible even if the mass changes associated with the modifications are similar. For example, embodiments of the present invention provide the ability to detect modifications that differ by about 0.036 amu, such as, acetyl and trimethyl modifications on a lysine amino acid. Advantageously, the applicability of embodiments of the present invention to the detection of protein modifications is not limited to a particular type of modification.

In embodiments of the present invention, SERS and Raman analysis can be used alone or in conjunction with mass spectrometry (for example, ESI (electrospray ionization) or MALDI (matrix-assisted laser desorption/ionization) mass spectrometry) to obtain protein modification information or protein profiles of different biomaterials for applications such as disease diagnosis and prognosis, and drug efficacy studies. Referring now to FIG. 1, a flow chart is provided generally outlining a method for protein profiling according to an embodiment of the present invention. Typically, a sample obtained from a biologic source, such as for example, a bodily fluid or cell lysate solution, is a complex mixture of proteins and other molecules. The components of the mixture can be separated using known techniques for isolating protein fractions from biologic samples, such as for example, physical or affinity based separation techniques. The isolated proteinaceous fraction can then be digested into smaller peptides. Typical methods include enzymatic digestions such as for example, proteinase enzymes such as, Arg-C (N-acetyl-gamma-glutamyl-phosphate reductase), Asp-N, Glu-C, Lys-C, chromotrypsin, clostripain, trypsin, and thermolysin. The resulting digest of peptides can be further separated, for example, using HPLC (high pressure liquid chromatography). Raman spectroscopy can then be performed on the resulting sample by, for example, mixing the digested sample with a SERS solution, such as for example, a colloidal silver solution, depositing and drying the digested sample onto a substrate and subsequently adding a SERS solution, such as a colloidal silver solution, depositing the sample onto a SERS-active substrate, or it can be performed in-line in a component of a microfluidic or nanofluidic system, such as by using a micro or nanomixer to mix the SERS solution with a the digested sample and subsequently performing Raman analysis on the sample. A silver colloidal solution can be mixed with digested sample eluants in a fluidic format (optionally, on a chip) and the detection can be performed inline as the eluants are flowing through the laser detection volume. In additional embodiments, some or all of these steps are performed using microfluidics.

In general, in embodiments of the invention, the detection target or biologic sample can be found in any type of animal or plant cell, or unicellular organism. For example, an animal cell could be a mammalian cell such as an immune cell, a cancer cell, a cell bearing a blood group antigen such as A, B, D, or an HLA antigen, or virus-infected cell. Further, the detection target could be from a microorganism, for example, bacterium, algae, virus, or protozoan. The analyte may be a molecule found directly in a sample such as a body fluid from a host. The body fluid can be, for example, urine, blood, plasma, serum, saliva, semen, stool, sputum, cerebral spinal fluid, tears, mucus, and the like.

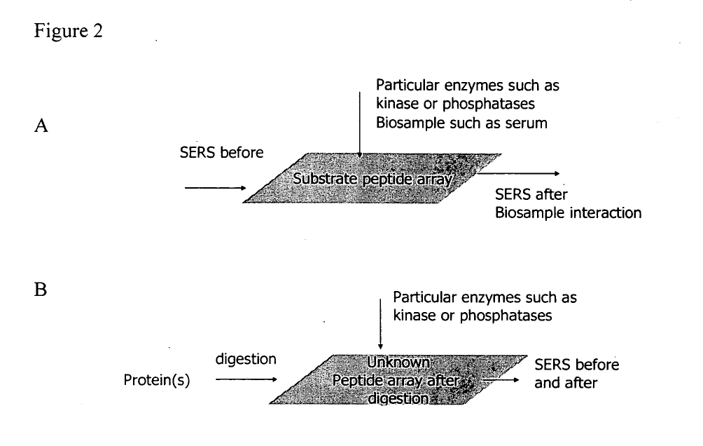

Raman surfaces of various forms can be used in embodiments of the present invention. For example, Raman active surfaces include, but are not limited to: a metallic surface, such as one or more layers of nanocrystalline and/or porous silicon coated with a metal or other conductive material; a particle, such as a metallic nanoparticle; an aggregate of particles, such as a metallic nanoparticle aggregate; a colloid of particles (with ionic compounds), such as a metallic nanoparticle colloid; or combinations thereof. Typical metals used for Raman enhancement include, silver, gold, platinum, copper, aluminum, or other conductive materials, although any metals capable of providing a SERS signal may be used. The particles or colloid surfaces can be of various shapes and sizes. In various embodiments of the invention, nanoparticles of between 1 nanometer (nm) and 2 micrometers (μm) in diameter may be used. In alternative embodiments of the invention, nanoparticles of 2 nm to 1 μm, 5 nm to 500 nm, 10 nm to 200 nm, 20 nm to 100 nm, 30 nm to 80 nm, 40 nm to 70 nm or 50 nm to 60 nm diameter may be used. In certain embodiments of the invention, nanoparticles with an average diameter of 10 to 50 nm, 50 to 100 nm or about 100 nm may be used. 10026] In additional embodiments of the present invention enzymatic activity assays, such as, for example, phosphotase, kinase, acetylase, and deacetylase assays, are performed using SERS spectroscopy. For example, FIG. 2 shows a schematic illustrating two exemplary methods for enzymatic activity profiling. In FIG. 2A, an array containing known peptides is synthesized using, for example, photolithography or spotting techniques, and is used as the substrate for testing the activity, such as for example detection or quantification of the activity of different types of enzymes, such as, for example, kinases, or phosphatases, or cell lysates or other samples of biologic origin. In a second example shown in FIG. 2B, the array is comprised of unknown peptides obtained from digestion of proteins. The array can be made, for example, by spotting the sample containing the digested material onto a substrate, using for example, a commercially available array spotter. The substrate, for example, is a silver or gold surface and the peptides are attached through metal-thiol linkages. Additionally, the substrate could be a porous silicon surface having a gold or silver layer. SERS is performed before and after the enzymatic or lysate activity on the substrate peptide array to determine the activity of particular enzymes on particular substrate peptides or lysates on particular peptides. In the case of peptides attached to a gold or silver surface, SERS is performed, for example, by depositing SERS active metal particles on the surface. The SERS particles can then be removed, for example by washing them from the surface, and the enzyme assay performed. SERS is then performed again by depositing SERS active metal particles once again on the substrate surface. In the case of the metal-coated porous silicon substrate, the substrate can act as an enhancement vehicle or SERS active metal particles can be deposited on the surface. The activity of particular enzymes is determined and profiles are generated from different biofluids.

Array compositions may include at least a surface with a plurality of discrete substrate sites. The size of the array will depend on the end use of the array. Arrays containing from about 2 to many millions of different discrete substrate sites can be made. Generally, the array will comprise from two to as many as a billion or more such sites, depending on the size of the surface. Thus, very high density, high density, moderate density, low density or very low density arrays can be made. Some ranges for very high-density arrays are from about 10,000,000 to about 2,000,000,000 sites per array. High-density arrays range from about 100,000 to about 10,000,000 sites. Moderate density arrays range from about 10,000 to about 50,000 sites. Low-density arrays are generally less than 10,000 sites. Very low-density arrays are less than 1,000 sites.

The sites comprise a pattern or a regular design or configuration, or can be randomly distributed. A regular pattern of sites can be used such that the sites can be addressed in an X-Y coordinate plane. The surface of the substrate can be modified to allow attachment of analytes at individual sites. Thus, the surface of the substrate can be modified such that discrete sites are formed. In one embodiment, the surface of the substrate can be modified to contain wells or depressions in the surface of the substrate. This can be done using a variety of known techniques, including, but not limited to, photolithography, stamping techniques, molding techniques and microetching techniques. As will be appreciated by those in the art, the technique used will depend on the composition and shape of the substrate.

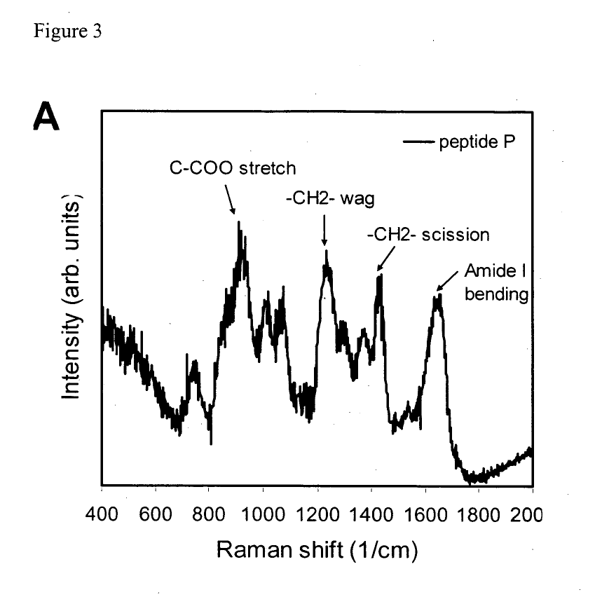

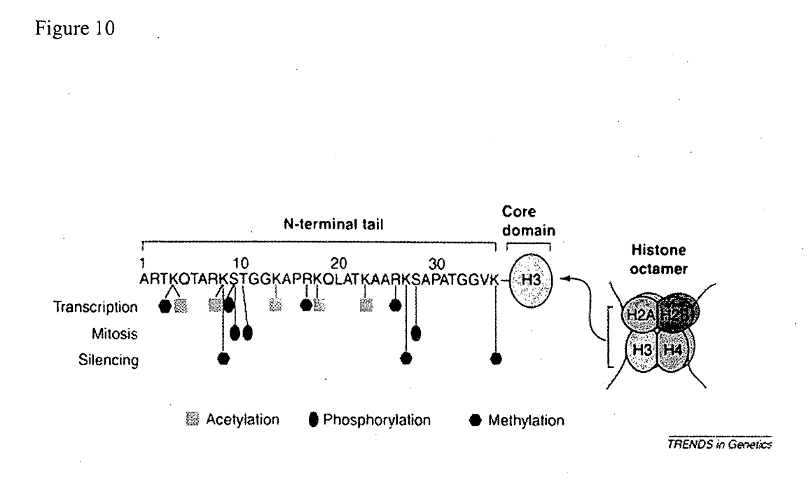

In additional embodiments, the present invention provides the ability to detect the presence of post-translational modifications of similar mass on peptides using SERS. For example, part of the N-terminal tail of histone H3 (⁹KSTGGKAPR) (P) has lysines at the amino-acid positions 9 and 14 that are frequently targeted for modifications such as acetylation and methylation. Similarly, the serine and threonine at amino acid positions 10 and 11 in this peptide, P, are targeted for phosphorylation. (See FIG. 10 for a map of biologically significant modification sites.) These modifications are known to have major effects on the histone-histone as well as the histone-regulatory protein interactions (see for example, S. K. Kurdistani, S. Tavazoie, M. Grunstein, Cell 117, 721-733 (2004); T. Kouzarides, Curr. Opin. Genet. Dev., 12, 198-209 (2002); B. D. Strahl, C. D. Allis, Nature 403, 41-45 (2000); S. J. Nowak, V. G. Corces, Trends in Genetics 20, 214-220 (2004); S. L. Berger, Curr. Opin. Genet. Dev., 12, 142-148 (2002); and Tamaru H. et al., Nat. Genet. 34, 75-79 (May 2003, 2003)). FIG. 3 shows the SERS spectrum of the unmodified peptide from the N-terminal tail of histone H3 (⁹KSTGGKAPR). The peaks in the SERS spectrum can be assigned to different vibrational bands within the peptide (see, for example, S. Stewart, P. M. Fredericks, Spectrochimica Acta Part A 55, 1615-1640 (1999); W. Herrebout, K. Clou, H. 0. Desseyn, N. Blaton, Spectrochimica Acta Part A 59, 47-59 (2003)). Particularly strong peaks can be observed at 919 cm⁻¹ (C—COO⁻), 1250 cm⁻¹ (CH₂ wag), 1436 cm⁻¹ (CH₂ scission) and 1655 cm⁻¹ (Amide I).

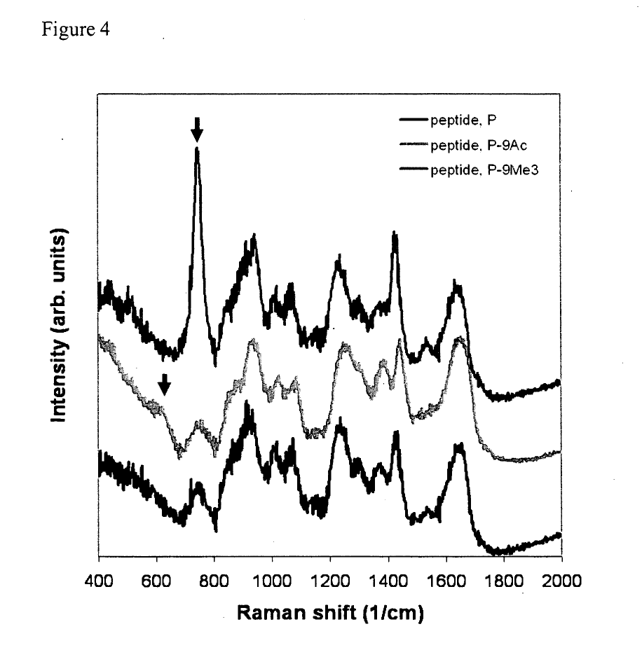

Referring now to FIG. 4, FIG. 4 compares the SERS spectra for the 9-trimethylated (P-9Me3) and 9-acetylated (P-9Ac) peptides to that of the corresponding unmodified peptide. The spectral signatures of the peptides differ based on the modification of a single amino acid. Peaks were observed in the SERS spectra of both the trimethylated and acetylated peptides that were absent from the spectrum of the unmodified peptide as indicated by the arrowheads in FIG. 4. As can be seen from FIG. 4, even though the mass difference between these modifications is only 0.03639 amu, they can be distinguished from one another. A very strong peak is observed at a wave-number of 744 cm⁻¹ for the 9-trimethylated peptide, P-9Me3, due to the trimethyl modification (CH₃ terminal rocking) of the lysine. The high signal intensity of this peak is believed to be attributed to the strong interaction between the positively charged N-terminus and the trimethyl ammonium side chain with the negatively charged silver nanoparticles (the surface charge density (Zeta potential) for the silver colloidal particles were measured using a Zetasizer (Zetasizer Nano, Malvern) and found to be about 62±3 mV). In the case of the 9-acetylated peptide, P-9Ac, a strong peak is observed at a wave-number of 628 cm⁻¹ that can be assigned to the side chain O═C—N bending resulting from the acetyl modification.

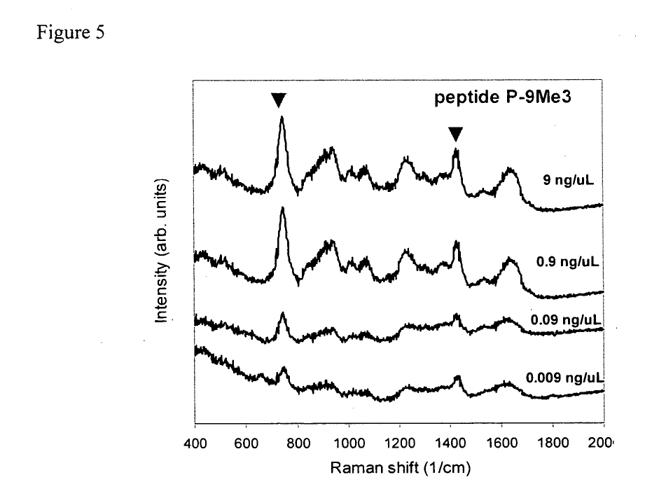

In an additional embodiment, using SERS, zeptomoles of the trimethylated modified peptide P-9Me3 were detected. This is useful because the stoichiometry of post-translational modifications can be very low. FIG. 5 shows the spectra of the 9-trimethylated peptide P-9Me3 at different concentrations over three orders of magnitude ranging from 9 ng/μl to 9 pg/μl. Concentrations down to 9 pg/μl, which corresponds to about 10 fmol/μl, exhibit the same features (strong peaks at 744 cm⁻¹ and 1436 cm⁻¹) observed in spectra from higher concentrations of the 9-trimethylated peptide P-9Me3. A concentration of 9 pg/μl corresponds to about 10 zeptomoles of the 9-trimethylated peptide P-9Me3 in the collection volume of the laser beam (the collection volume of the laser illumination spot was estimated to be about 2.5 μm ×2.5 μm ×200 μm).

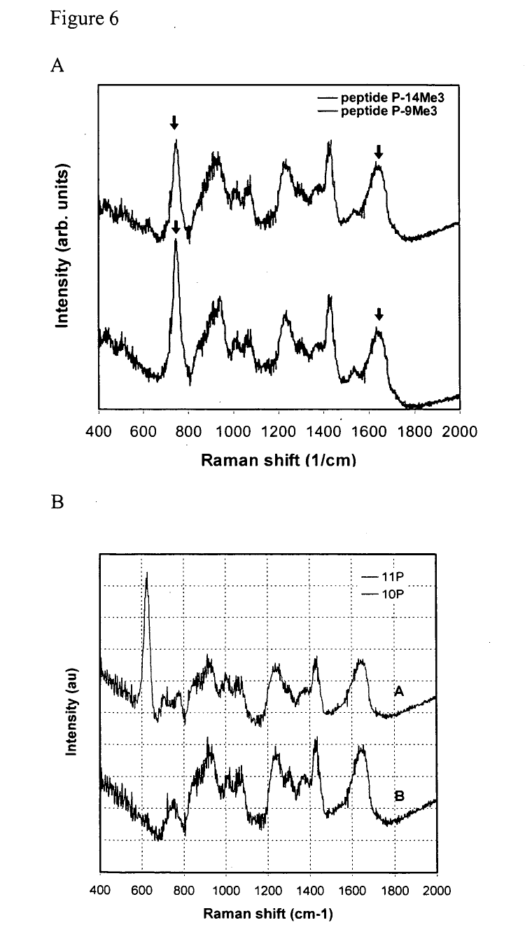

Embodiments of the present invention also provide methods for obtaining information for labile modifications such as, for example, serine and threonine phosphorylation. Referring now to FIG. 6, SERS was used to obtain positional information for trimethylation and phosphorylation modifications within a peptide. FIG. 6A compares the SERS spectra of a trimethylated modified peptide with the trimethylation modification at either the lysine at the 9 amino-acid position (P-9Me3) or at the lysine at the 14 amino-acid position (P-14Me3). It is apparent from the SERS spectra that the intensity of the peak at 744cm⁻¹ is reduced in the peptide P-14Me3 compared to the peptide P-9Me3 while the intensity of the peak at 1655 cm⁻¹, does not change significantly in the peptides. This is believed to be because the mechanism of SERS enhancement is attributed to both electromagnetic and chemical effects wherein chemical interactions between the molecules and the metal surfaces not only increase the scattering cross-section of the molecules but also provide the distinct advantage of discerning subtle chemical and conformational changes of molecules.

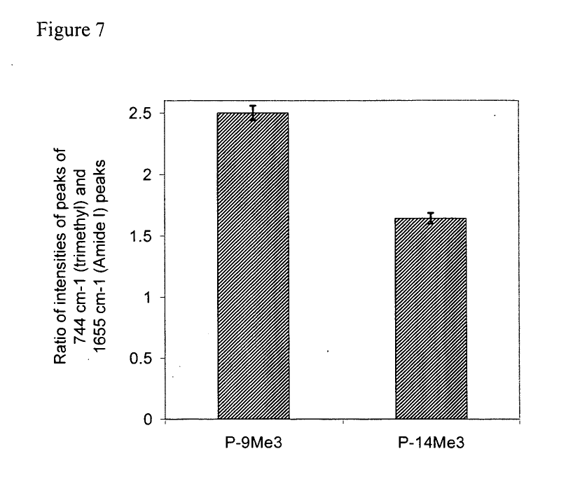

It is believed that adsorption and orientation of the molecules onto the silver nanoparticles also play a role in the SERS enhancement. Since the surface of the silver colloidal nanoparticles used in the SERS examples is negatively charged, it is likely that both the positively charged N-terminus of the peptide and the trimethyl modification adsorb to the silver nanoparticle surface. Consequently, in the case of the peptide P-9Me3 where the trimethyl modification moiety remains close to the metal surface, the peak at 744 cm⁻¹ is strongly enhanced. Whereas, in the peptide P-14Me3, where the trimethyl modification moiety is further away from the silver surface, the intensity of the peak at 744 cm⁻¹ drops relative to the other peaks in the spectra as can be seen in FIG. 7 in which the ratio of the intensities of peak corresponding to the trimethyl modification (at 744 cm⁻⁻) and Amide I (at 1655 cm⁻¹) is plotted for peptides P-9Me3 and P-14Me3. Data analysis was performed using 50 spectra with accumulation times of 1 s for each peptide.

In further embodiments, SERS is used for the detection and analysis of labile post translational modifications, such as, for example, phosphorylation. While the relative ratio of peaks is altered by trimethylation at different positions as shown in FIG. 6A, phosphorylation at different amino acid positions is marked by spectral signature changes. FIG. 6B illustrates the spectral differences between peptides phosphorylated at serine-10 (peptide P-10P, ⁹K¹⁰S_{PO3}TGGKAPR) and threonine-11 (peptide 11-P, ⁹KS ¹¹ T_{PO3}GGKAPR). A strong peak at 628 cm⁻¹ is present only in the case of the peptide P-11P and not in the peptide P-10P. It should be noted that these results were obtained from phosphorylated peptides obtained from a single supplier source. In the case of phosphorylation modification, the spectral differences are likely due to the negatively charged phosphate groups affecting the adsorption and orientation of the peptides onto the silver nanoparticles.

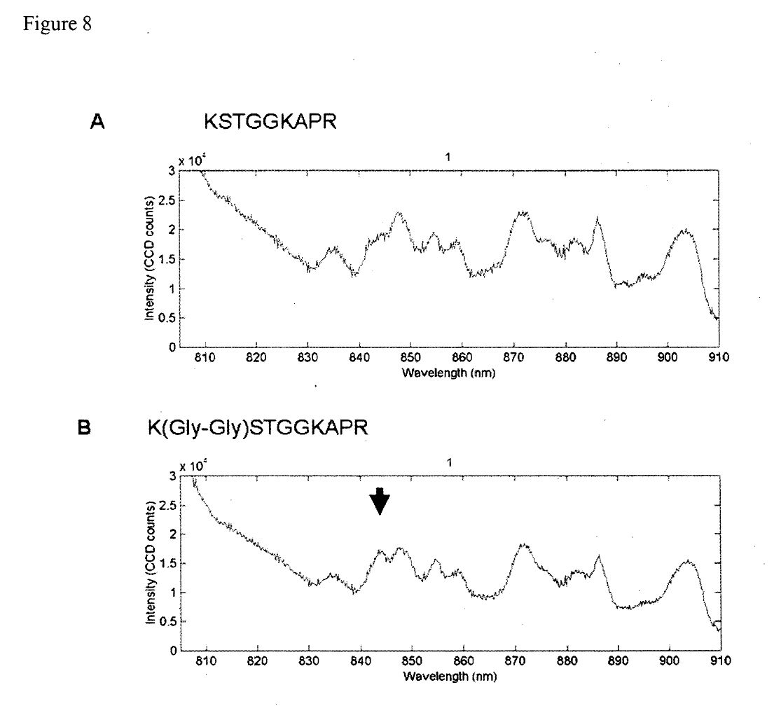

Additionally, FIGS. 8A and 8B illustrate the use of SERS to detect a ubiquination peptide modification. FIG. 8A provides a SERS spectrum of an unmodified peptide (⁹KSTGGKAPR) and FIG. 8B provides a corresponding SERS spectrum of a peptide ubiquitin analog (⁹K(Gly-Gly)STGGKAPR). An arrow in FIG. 8B indicates an important spectral difference between the unmodified peptide and the ubiquitin analog.

It was found that factors, such as, for example, the addition sequence of the SERS cocktail and the incubation time on the SERS spectra of a modified peptide such as, the acetylated peptide (K(Acetylated)STGGKAPR), affected the intensity of the spectrum obtained. Additionally, the pH, ionic strength, and surface properties of the SERS substrate affect the spectrum obtained. In some embodiments of the present invention, the pH was controlled to have a delta less than about 0.5 pH and ionic strength was controlled, for example, about 20-300. In addition to the potential effects of pH changes on the spectroscopic and biochemical measurements, the effects of buffering capacity, which are dependent on the concentrations and the types of buffers, also play a role in determining the spectra obtained. For example, performing SERS in acidic condition (such as directly from an HPLC eluent of 0.1% TFA in ACN) increases the signal variations from chemical bonds that are closer to the N-terminal; while performing SERS using Ag particles coated with hydrophobic compounds (such as alkyl-thiol) magnifies the signal change from hydrophobic amino acid such as tyrosine. Also, the use of complexing agents such as divalent salts (Ca²⁺) for masking or complexing with negative charges on a phosphorylation modification can help in bringing the biomolecule closer to the SERS substrate thereby increasing the ability to distinguish the modified peptide from an unmodified one.

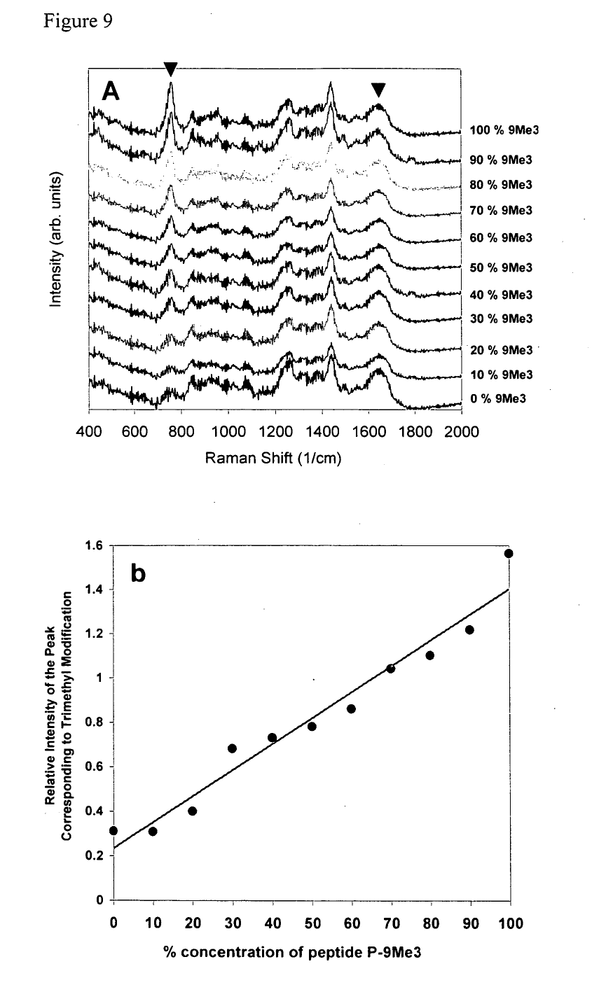

In additional embodiments, SERS is used to quantify the concentrations of peptides having different modifications in a mixture. For example, FIG. 9A shows the SERS spectra of a mixture of 9-dimethylated peptide, P-9Me2 (⁹K_{Me2}STGGKAPR) and 9-trimethylated peptide, P-9Me3 (⁹K_{Me3}STGGKAPR). The unique peak at 744 cm⁻¹ corresponding to the trimethylation modification from peptide P-9Me3 is visible in the spectra of the mixture. We performed quantification of trimethylation modification within the mixture using the SERS spectral information. SERS was performed on mixtures of different concentrations of 9-dimethylated and 9-trimethylated peptides, P-9Me2 and P-9Me3. FIG. 9B shows the graph of the ratio of the intensities at 744 cm⁻¹ (corresponding to the trimethyl modification) and at 1655 cm⁻¹ (corresponding to Amide I bending) plotted against % concentration of 9-trimethylated peptide P-9Me3. The linear trend for concentration versus peak intensity allows quantification of peptide concentrations in a sample by, for example, mapping peak intensity on a plot of known concentration versus peak intensity. This quantification ability allows, for example, enzymatic activity assays to be performed.

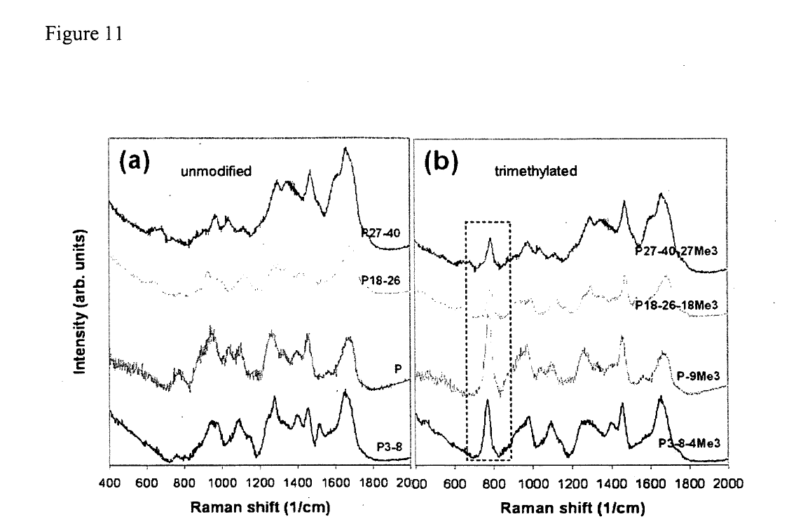

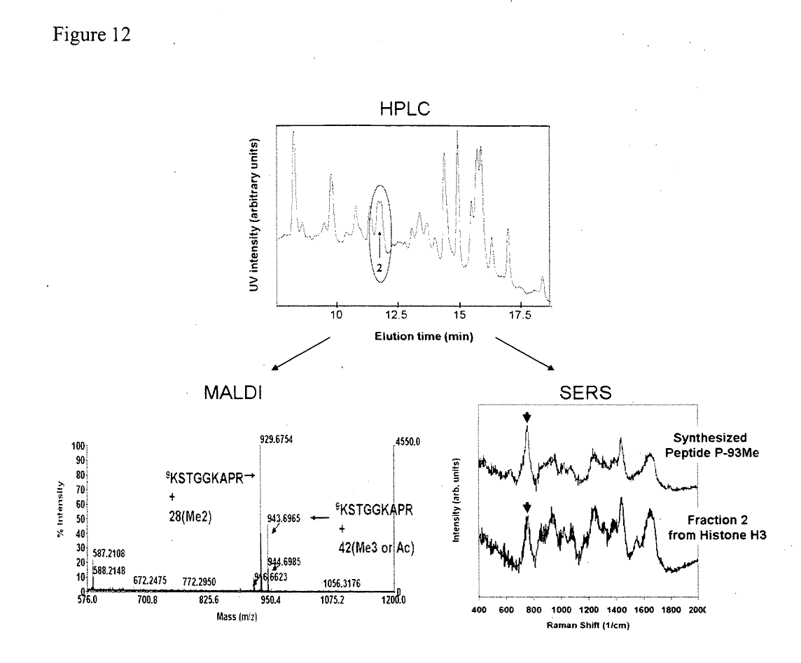

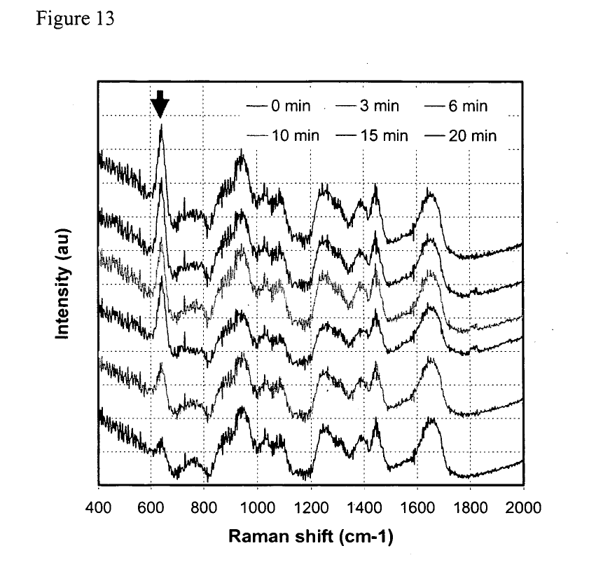

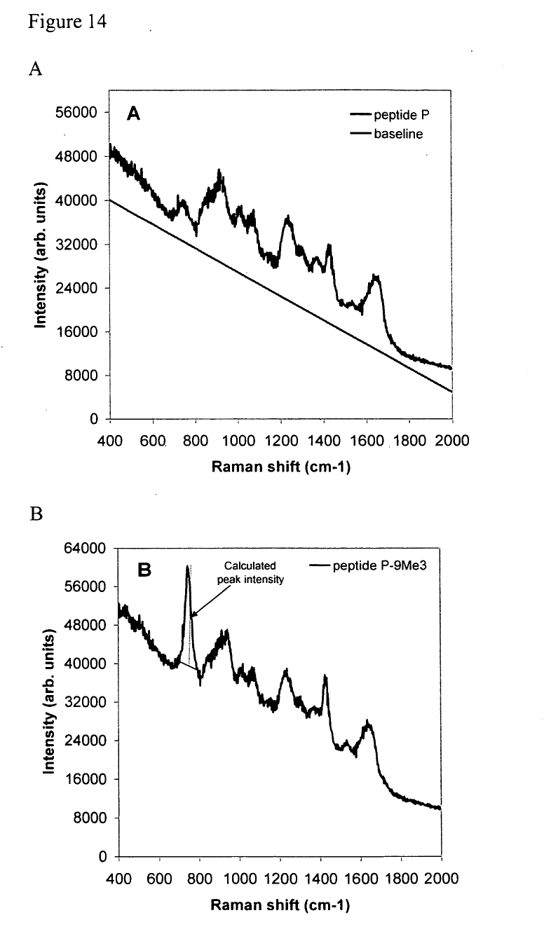

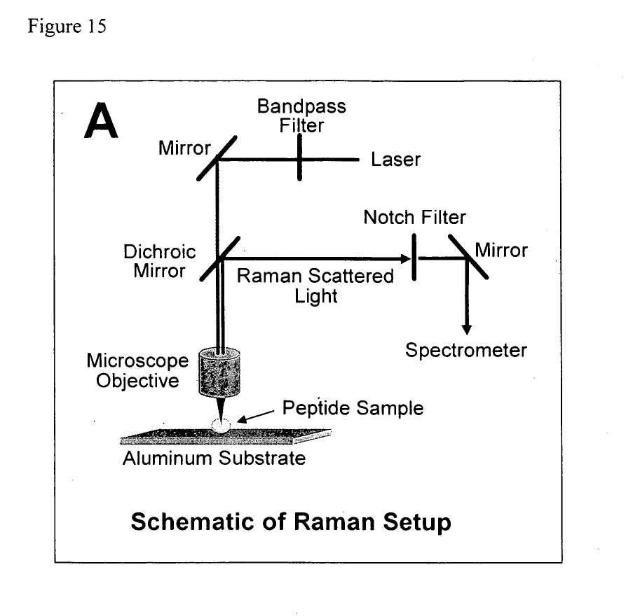

FIG. 10 maps the N-terminal tail of Histone H3. The biological significance of certain modifications in cellular functions such as, transcription, mitosis, and gene silencing, is indicated. FIG. 11A provides a comparison of SERS spectra obtained from different peptides from the N-terminal tail of Histone H3 and FIG. 11B provides a comparison of the corresponding trimethyl derivatives. The sequences for the unmodified peptides shown are: ³TKQTAR for the spectrum labeled P3-8, ⁹KSTGGKAPR for the spectrum labeled P, ¹⁸KQLATKAAR for the spectrum labeled P18-26, and ²⁷KSAPSTGGVKKPHR for the spectrum labeled P27-40. The sequences for the trimethylated peptides shown are: ³TK(trimethyl)QTAR for the spectrum labeled P3-8-4Me3, ⁹KSTGGKAPR for the spectrum labeled P, ¹⁸K(trimethyl)QLATKAAR for the spectrum labeled P18-26-18Me3, and ²⁷K(trimethyl)SAPSTGGVKKPHR for the spectrum labeled P27-40-27Me3. It can be seen from FIG. 11B that all the trimethylated peptides exhibit a characteristic peak at 744 cm⁻¹ irrespective of peptide sequence. This strong characteristic peak can be attributed to the terminal rocking of the methyl group.

In an additional example, we have used SERS as a complementary technique to mass spectrometry to identify and distinguish post translational modifications of similar mass, such as trimethylation and acetylation. Referring now to FIG. 12, FIG. 12A shows an HPLC chromatogram of digested Histone H3 isolated from calf thymus using a C18 column indicating the fraction (fraction 2) that was collected and analyzed using MALDI-TOF and SERS techniques. Histone H3 was digested with Arg-C endoproteinase, separated by reverse-phase liquid chromatography and the fractionated peptides were analyzed by SERS and MALDI-TOF. SERS in combination with MALDI helped distinguish trimethylation versus acetylation of Lys9 of the N-terminal tail of Histone H3. FIG. 12B shows the MALDI-TOF spectrum obtained from fraction 2 of the HPLC chromatogram of FIG. 12A. As can be seen from the spectrum in FIG. 12B, fraction 2 contained a mixture of peptides having masses of 929.67 Da and 943.69 Da. The peak at mass 929.67 Da corresponds to a mass difference of +28 Da from peptide P, KSTGGKAPR, and is the dimethylated peptide, P-9Me2 (from MS/MS (Tandem Mass Spectrometry) measurements). The peak at mass 943.69 Da corresponds to a modification having a +42 Da mass difference at Lys9 of peptide P (from MS/MS measurements). This mass difference could be due either to acylation or trimethylation. FIG. 12C presents a comparison of the SERS spectrum obtained from fraction 2 from the digested and separated Histone H3 and synthesized trimethylated peptide, P-9Me3. The SERS spectra from this fraction obtained from the digested histone when compared with the spectra from the synthesized peptide, P-9Me3, shows a clear peak corresponding to trimethylation at 744 cm⁻¹ indicating that the peptide is trimethylated and not acetylated at Lys9. Thus, SERS is a powerful complementary techniques to mass spectroscopy in distinguishing similar mass modifications.

A non-limiting example of a Raman detection unit is disclosed in U.S. Pat. No. 6,002,471. An excitation beam is generated by either a frequency doubled Nd:YAG laser at 532 nm wavelength or a frequency doubled Ti:sapphire laser at 365 nm wavelength. Pulsed laser beams or continuous laser beams can be used. The excitation beam passes through confocal optics and a microscope objective, and is focused onto the flow path and/or the flow-through cell. The Raman emission light is collected by the microscope objective and the confocal optics and is coupled to a monochromator for spectral dissociation. The confocal optics includes a combination of dichroic filters, barrier filters, confocal pinholes, lenses, and mirrors for reducing the background signal. Standard full field optics can be used as well as confocal optics. The Raman emission signal is detected by a Raman detector that includes an avalanche photodiode interfaced with a computer for counting and digitization of the signal.

Another example of a Raman detection unit is disclosed in U.S. Pat. No. 5,306,403, including a Spex Model 1403 double-grating spectrophotometer with a gallium-arsenide photomultiplier tube (RCA Model C31034 or Burle Industries Model C3103402) operated in the single-photon counting mode. The excitation source includes a 514.5 nm line argon-ion laser from SpectraPhysics, Model 166, and a 647.1 nm line of a krypton-ion laser (Innova 70, Coherent).

Alternative excitation sources include a nitrogen laser (Laser Science Inc.) at 337 nm and a helium-cadmium laser (Liconox) at 325 nm (U.S. Pat. No. 6,174,677), a light emitting diode, an Nd:YLF laser, and/or various ions lasers and/or dye lasers. The excitation beam can be spectrally purified with a bandpass filter (Corion) and can be focused on the flow path and/or flow-through cell using a 6X objective lens (Newport, Model L6X). The objective lens can be used to both excite the Raman-active probe constructs and to collect the Raman signal, by using a holographic beam splitter (Kaiser Optical Systems, Inc., Model HB 647-26N18) to produce a right-angle geometry for the excitation beam and the emitted Raman signal. A holographic notch filter (Kaiser Optical Systems, Inc.) can be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors can be used, such as Fourier-transform spectrographs (based on Michaelson interferometers), charged injection devices, photodiode arrays, InGaAs detectors, electron-multiplied CCD, intensified CCD and/or phototransistor arrays.

In certain aspects of the invention, a system for detecting the target complex of the present invention includes an information processing system. An exemplary information processing system may incorporate a computer that includes a bus for communicating information and a processor for processing information. The information processing and control system may further comprise any peripheral devices known in the art, such as memory, display, keyboard and/or other devices.

While certain methods of the present invention can be performed under the control of a programmed processor, in alternative embodiments of the invention, the methods can be fully or partially implemented by any programmable or hardcoded logic, such as Field Programmable Gate Arrays (FPGAs), TTL logic, or Application Specific Integrated Circuits (ASICs). Additionally, the disclosed methods can be performed by any combination of programmed general purpose computer components and/or custom hardware components.

Following the data gathering operation, the data is typically reported to a data analysis operation. To facilitate the analysis operation, the data obtained by the detection unit will typically be analyzed using a digital computer such as that described above. Typically, the computer will be appropriately programmed for receipt and storage of the data from the detection unit as well as for analysis and reporting of the data gathered.

In certain embodiments of the invention, custom designed software packages can be used to analyze the data obtained from the detection unit. In alternative embodiments of the invention, data analysis can be performed using an information processing system and publicly available software packages.

### EXAMPLE 1

SERS experiments were performed as follows.

Colloidal Silver Preparation

Colloidal silver suspension was prepared by citrate reduction of silver nitrate as described in Lee and Meisel (P. C. Lee, D. J. Meisel, Phys. Chem. 86, 3391 (1982)). The suspension had a final silver concentration of 1.00 mM. The surface charge density (Zeta potential) for the colloidal silver particles, after diluting 20 times with deionized (DI) water, was found to be 62±3 mV using a Zetasizer (Zetasizer Nano, Malvern).

Peptide Synthesis

Peptides with and without modifications were synthesized using Solid Phase Peptide Synthesis (SPPS) methods with standard Fmoc/t-buty/trityl protection chemistries to build up a full-length peptide chain. The starting amino acid was bound to a solid resin support (usually polystyrene) and its alpha amino group was chemically “blocked” with the Fmoc protecting group. Reactive side-chains were blocked with either t-Butyl or Trityl groups. The alpha-amino Fmoc protecting group was removed and an incoming amino acid (which was chemically activated on its carboxyl terminus to form an active ester) condensed to form a peptide bond. The process was repeated until the full-length product was obtained. The resin-bound peptide was then treated with trifluoroacetic acid (TFA) to remove the side-chain protecting groups and cleave the peptide from the polystyrene resin. Peptides were then precipitated out of solution with MTBE (methyl tertiary butyl ether) and lyophilized to dryness. For synthesis of modified peptides, trimethylated amino acid analogs were bought from Bachem in Switzerland, phospho-amino acids and acetyl-lysine were purchased from Nova Biochem in San Diego, Calif. Reverse-phase HPLC was utilized to purify and separate the target peptide from a crude mixture. MALDI-TOF mass spectrometry was used to determine the peptide's mass and compare with the expected peptide mass to confirm fidelity of the synthesis and purity of the product.

SERS Measurements

Peptides lyophilized after synthesis were resuspended in DI water at a concentration of 1 μg/μl and diluted to various sample concentrations. The stock solution of the synthesized colloidal silver, with a final silver concentration of 1.00 mM, was diluted 1 part to 2 parts in volume of DI water. Typically, 10 μl of the peptide solution was incubated with 80 μl of the diluted silver solution for 15 min. 20 μl of 0.5 M LiCl solution was added after the incubation and the solution was mixed thoroughly and dropped onto an aluminum tray for immediate SERS measurements. The laser was focused inside the sample droplet and 50 - 100 spectra were collected for each peptide sample. Typical collection time of each spectrum was 1 sec. A raw sample spectrum of the unmodified peptide P is shown in FIG. 14A. Background from the spectra was subtracted by fitting an arbitrary linear baseline (also shown in FIG. 14A). Intensities of the peaks were calculated directly from the raw spectra by calculating the distance between the apex of the peak area and the midpoint of the base points of the peak area (FIG. 14B).

FIG. 13 provides SERS spectra of peptide P-9Ac at different incubation times of the silver nanoparticles with the sample. In this example, 80 μl of silver solution (1:2 diluted in water) was mixed with 10 μl of the peptide (100 ng/μl) and incubated at room temperature for between 0-20 min. Then, 20 μl of lithium chloride solution (0.5 M in DI water) was added to the above solution and SERS spectra were accumulated immediately after LiCl addition by dropping the solution onto an aluminum substrate. In the case of the 9-acetylated peptide, P-9Ac, a strong peak is observed at a wave-number of 628 cm⁻¹ and it was found that the intensity of this peak was dependent on the incubation time of the sample with the silver nanoparticles before the addition of lithium chloride for aggregation.

FIG. 15 shows a schematic of a Raman spectrometer setup that was used for the SERS measurements. The system consisted of a titanium:sapphire laser 10 (Mira by Coherent, Santa Clara, Calif.) operating at 785 nm with power levels of about 750 mW, and a 20X microscope objective 20 (Nikon LU series) to focus the laser spot onto the sample plane. The peptide sample 30 was placed on an aluminum substrate 40. The excitation beam 50 was filtered by a dielectric filter 60 (Chroma Technology Corp., Brattleboro, Vt.), to suppress spontaneous emission from the laser and transmitted through a dichroic mirror 60 (Chroma Technology Corp., Brattleboro, Vt.). The Raman scattered light from the sample 70 was collected by the same microscope objective 20, and was reflected off the dichroic mirror 60 toward a notch filter or bandpass filter 80 (Kaiser Optical Systems, Ann Arbor, Mich.). The notch filter blocked the laser beam and transmitted Raman scattered light. The Raman-scattered light was imaged onto the slit of a spectrophotometer 90 (Acton Research Corp., Acton, Mass.) connected to a thermo-electrically cooled charge-coupled device (CCD) detector (Princeton Instruments, Princeton, N.J.) (not shown). The CCD camera was connected to a PC (not shown), and the collected spectrum was transported to the PC for visual display and computational analysis.

### EXAMPLE 2

The detection of post-translational modifications from biological samples was performed as follows.

Enzymatic Digestion of Histone H3

Lyophilized Histone H3 (obtained from Roche Applied Science, Inc.) was reconstituted in DI water to a concentration of 5 μg/μl. 5 μl of the reconstituted Histone H3 was digested with 250 ng of Endoproteinase Arg-C (enzyme substrate ration of 1:100 in a total volume of 50 μl of 50 mM ammonium bicarbonate buffer. Digestions were carried out at 37° C. for 16 hours. Digestion was halted by adding trifluoroacetic acid (TFA) to the digestion mixture at a final concentration of 0.5%.

HPLC Separation of Digested Histone H3

HPLC separation of the peptides from the digested Histone H3 was performed using an Alltech C18 column (150 mm×4.6 mm) using a two-step gradient. The gradients increased from 2 to 65% B over 63 min., stayed at 65% B for 7 min., and then increased from 65 to 85% B over 5 min. Solution A was 0.1% TFA in water and Solution B was 0.065% TFA in acetonitrile. Detection wavelength was 210 nm. Flow rate was 500 μl/min. Fractions were collected using an automated fraction collector every 10 s and combined according to peak positions and elution time. The combined fractions were then lyophilized to get rid of the mobile phase and then resuspended in 5 μl DI water for subsequent SERS and MALDI-TOF experiments.

SERS Measurements

Peptides lyophilized after synthesis and HPLC fraction collection were resuspended in DI water and diluted to various sample concentrations. The stock solution of the synthesized colloidal silver, with a final silver concentration of 1.00 mM, was diluted 1 part to 2 parts in volume of DI water. Typically, 10 μl of the peptide solution was incubated with 80 μl of the diluted silver solution at room temperature for 15 min. 20 μl of 0.5 M LiCi solution was added after the incubation and the solution was mixed thoroughly and dropped onto an aluminum plate for immediate SERS measurements. The laser was focused inside the sample droplet and 50 - 100 spectra were collected for each peptide sample. Typical collection time of each spectrum was 1 sec. Background from the spectra was subtracted by fitting an arbitrary linear baseline (shown in FIG. 14A). Intensities of the peaks were calculated directly from the raw spectra by calculating the distance between the apex of the peak area and the midpoint of the base points of the peak area (FIG. 14B).

SERS measurements were performed as on a Raman spectrometer described in Example 1 and FIG. 15.

Maldi-TOF Measurements

Samples were spotted onto a target and MALDI data were collected on a Voyager DE-Pro mass spectrometer (Applied Biosystems) operated in reflection mode and calibrated externally.

What is claimed is:

1) A method for detecting a modification state of a peptide or protein comprising,
obtaining a sample containing a target peptide or protein,
isolating a proteinaceous fraction from the sample containing the target peptide or protein,
fragmenting proteinaceous material in the proteinaceous fraction to create smaller peptides,
obtaining a Surface Enhanced Raman Spectrum (SERS) of one or more of the smaller peptides, and
determining a modification state of at least one smaller peptide from the data contained in the Surface Enhanced Raman Spectrum.

2) The method of claim 1 additionally comprising obtaining a mass spectrum of the smaller peptides.

3) The method of claim 1 wherein fragmenting comprises digesting the proteinaceous fraction with a proteinase enzyme.

4) The method of claim 1 wherein obtaining the Surface Enhanced Raman spectrum comprises adsorbing one or more of the smaller peptides onto a Surface Enhanced Raman active substrate.

5) The method of claim 4 wherein the Surface Enhanced Raman active substrate comprises a metallic substrate surface, a metallic particle, an aggregate of metallic particles, a colloid of metallic particles, or a combination thereof.

6) The method of claim 4 wherein the Surface Enhanced Raman active substrate comprises silver or gold.

7) The method of claims 5 or 6 wherein the Surface Enhanced Raman active substrate also comprises lithium chloride.

8) The method of claim 1 wherein the modification state of the peptide comprises dimethylation, trimethylation, acetylation, phosphorylation, ubiquitination, glycosylation, nitrosylation, lipidation, palmitoylation, or a combination thereof.

9) The method of claim 1 wherein the modification state of the peptide comprises dimethylation, trimethylation, or acetylation.

10) A method for quantifying the amount of modified peptide or protein in a sample comprising,
obtaining a sample containing a target peptide or protein,
isolating a proteinaceous fraction from the sample containing the target peptide or protein,
fragmenting proteinaceous material in the proteinaceous fraction to create smaller peptides,
obtaining a Surface Enhanced Raman Spectrum of one or more of the smaller peptides, and
comparing one or more peak intensities from the Enhanced Raman Spectrum to a peak intensity from a sample containing a known amount of smaller peptide to determine the amount of peptide from the sample.

11) The method of claim 10 wherein fragmenting comprises digesting the proteinaceous fraction with a proteinase enzyme.

12) The method of claim 10 wherein obtaining the Surface Enhanced Raman spectrum comprises adsorbing one or more of the smaller peptides onto a Surface Enhanced Raman active substrate.

13) The method of claim 12 wherein the Surface Enhanced Raman active substrate comprises a metallic substrate surface, a metallic particle, an aggregate of metallic particles, a colloid of metallic particles, or a combination thereof.

14) The method of claim 12 wherein the Surface Enhanced Raman active substrate comprises silver or gold.

15) The method of claims 13 or 14 wherein the Surface Enhanced Raman active substrate also comprises lithium chloride.

16) The method of claim 10 wherein the modification state of the peptide comprises dimethylation, trimethylation, acetylation, phosphorylation, ubiquitination, glycosylation, nitrosylation, lipidation, palmitoylation, or a combination thereof.

17) The method of claim 10 wherein the modification state of the peptide comprises dimethylation, trimethylation, or acetylation.

18) A method for analyzing a sample comprising:
providing a substrate having a surface and a plurality of peptides attached to the surface,
analyzing the surface using Surface Enhanced Raman Spectroscopy,
contacting the substrate surface with a fluid sample under conditions that allow any components of the sample that are capable of interacting with the plurality of peptides attached to the substrate to react with the peptides attached to the substrate,
analyzing the surface of the substrate an additional time using Surface Enhanced Raman Spectroscopy, and
determining a modification state of at least one peptide from the data contained in a Raman spectrum.

19) The method of claim 18 wherein the sample is a biofluid.

20) The method of claim 18 wherein the sample contains an enzyme selected from the group consisting of phosphotase, kinase, acetylase, and deacetylase.

21) The method of claim 18 wherein the plurality of peptides form an array of peptides.

22) The method of claim 18 wherein the surface is a Raman active surface comprised of gold or silver.

23) The method of claim 18 wherein the surface is a Raman active surface comprised of porous silicon coated with gold or silver.

24) The method of claim 22 or 23 wherein the Raman active surface also comprises lithium chloride.

25) The method of claim 18 wherein analyzing the surface using Surface Enhanced Raman Spectroscopy includes depositing Surface Enhanced Raman active metal particles on the surface.

26) The method of claim 25 wherein the Raman active metal particles are nanoparticles comprised of silver or gold.

27) The method of claim 26 wherein the Raman active metal nanoparticles are activated with lithium chloride.


SEQUENCE LISTING (11 sequences)

SEQ ID NO: 1 - protein, 9 aa, Drosophila
KSTGGKAPR

SEQ ID NO: 2 - protein, 9 aa, Drosophila
  MOD_RES (1)..(1): tri-METHYLATION
KSTGGKAPR

SEQ ID NO: 3 - protein, 9 aa, Drosophila
  MOD_RES (1)..(1): ACETYLATION
KSTGGKAPR

SEQ ID NO: 4 - protein, 9 aa, Drosophila
  MOD_RES (6)..(6): tri-METHYLATION
KSTGGKAPR

SEQ ID NO: 5 - protein, 9 aa, Drosophila
  MOD_RES (3)..(3): PHOSPHORYLATION
KSTGGKAPR

SEQ ID NO: 6 - protein, 9 aa, Drosophila
  MOD_RES (2)..(2): PHOSPHORYLATION
KSTGGKAPR

SEQ ID NO: 7 - protein, 9 aa, Drosophila
  MOD_RES (1)..(1): gly-gly
KSTGGKAPR

SEQ ID NO: 8 - protein, 9 aa, Drosophila
  MOD_RES (1)..(1): di-METHYLATION
KSTGGKAPR

SEQ ID NO: 9 - protein, 6 aa, Drosophila
TKQTAR

SEQ ID NO: 10 - protein, 9 aa, Drosophila
KQLATKAAR

SEQ ID NO: 11 - protein, 14 aa, Drosophila
KSAPSTGGVKKPHR